(19) United States
(12) Patent Application Publication
(10) Pub. No.: US 2023/0058803 A1
(43) Pub. Date: Feb. 23, 2023

(54) METHOD, DEVICE, AND SYSTEM FOR REMOTELY MONITORING A MEDICAL DEVICE

(71) Applicant: FRESENIUS MEDICAL CARE DEUTSCHLAND GMBH, Bad Homburg (DE)

(72) Inventor: Lars OFFERMANNS, Butzbach (DE)

(73) Assignee: FRESENIUS MEDICAL CARE DEUTSCHLAND GMBH, Bad Homburg (DE)

(21) Appl. No.: 17/796,686

(22) PCT Filed: Feb. 9, 2021
(86) PCT No.: PCT/EP2021/053007

§ 371 Date: Aug. 1, 2022

(30) Foreign Application Priority Data
Feb. 10, 2020 (DE) 10 2020 201 627.5

(51) Int. Cl. G16H 40/60

(52) U.S. Cl. CPC G16H 40/60; G16H 40/67

(57) ABSTRACT

The invention relates to a method and a system for the remote monitoring of a medical technical apparatus used, in particular in a closed patient treatment room, for the medical monitoring or treatment of a specific patient, in particular from outside the patient treatment room. The method comprises: detection, in a sensor-based manner, of a machine-readable patient identification assigned to the patient; initiation of a data coupling between the medical technical apparatus and the terminal apparatus on the basis of the detected patient identification in order to establish a wireless communication link for the exchange of data assigned to the patient identification between the medical technical apparatus and the terminal apparatus; reception, via the communication link which has been established, of information transmitted by the medical technical apparatus to the terminal apparatus relating to the patient identified on the basis of the patient identification, or relating to their monitoring or treatment by the medical technical apparatus; and output of a representation of the received information at a human-machine interface of the terminal apparatus. The invention further relates to a computer program configured to carry out the method, as well as a terminal apparatus, set up in a corresponding manner, as part of the system.

The present invention relates to the remote monitoring of a medical technical apparatus by means of a device which is separate from the medical technical apparatus and constructed as a mobile terminal apparatus. The invention is directed in particular to a method for the remote monitoring of a medical technical apparatus which is used in a patient treatment room, in particular in a closed patient treatment room, for the medical monitoring or treatment of a particular patient, in particular from outside the patient treatment room, by means of a mobile terminal apparatus which is separate from the medical technical apparatus. Furthermore, the invention is directed to a computer program, a terminal apparatus and a system, each of which is configured to carry out the method.

These days, a large number of medical technical apparatuses, in particular also electronic apparatuses, for monitoring and/or treating patients, in particular in the hospital environment, are known. In particular, these include apparatuses belonging to class A61M of the CPC patent classification, such as dialysis apparatuses, blood purification apparatuses, as well as monitoring or treatment apparatuses that are specifically provided for intensive medical care use. As a rule, such medical technical apparatuses comprise a user interface for displaying information as well as for their operation by doctors or health care personnel. Such user interfaces typically comprise at least one display device as well as one or more operating elements, such as for example switches or controls. Touch-sensitive screens (Touchscreens) are also often used in this day and age. Typically, these user interfaces are part of the medical technical apparatus itself and are provided on the apparatus itself.

In some situations it is necessary for a patient who is to be given medical care to be placed in a specially protected hygienic environment such as for example an intensive care unit or a quarantine ward, either in order to protect the patient himself or herself from potentially dangerous germs or contamination or, conversely, to minimize the risk of infection for other people emanating from the patient. An example of this is the risk of infection from multi-resistant germs.

In order to check, in particular to monitor, to configure or to control or otherwise to operate a medical technical apparatus used to monitor or treat such a patient, medical personnel must, as a rule, wear additional protective clothing, which must typically be put on before entering the patient treatment room in which the patient is located, and disposed of before leaving the room. Such protective clothing consists, as a rule, of a disposable hood, a disposable mask, a disposable gown, and disposable gloves. In addition, before entering and leaving the patient treatment room, hygienic hand disinfection must usually also be carried out. These complex hygiene procedures become necessary every time a check of the medical technical apparatus via its user interface is required, even if, for example, only a short reading of a display or a simple acknowledgement of a message needs to be carried out, which would require only a small amount of time without the hygiene measures. If there are several medical technical apparatuses in the patient treatment room which are to be supervised by the medical personnel, which may for example be the case if several patients are being supervised in the room, each of whom is being monitored or treated by a different medical technical apparatus, the personnel must also go to each of these apparatuses individually in order to check it via the respective user interface of the respective apparatus. This also leads to an increased supervision burden.

Overall, in such a particularly hygiene-sensitive environment, a high level of hygiene and a significant amount of time is thus required to check one or more medical technical apparatuses.

In such a hygiene-sensitive environment and also in situations in which no such special hygiene protection measures are required, in particular also in the case of non-enclosed patient treatment rooms, the operation of medical technical apparatuses for monitoring or treating patients may, in the course of their use, in particular the supervision of these apparatuses by medical personnel during their use, require longer walking distances for the medical personnel, which may be time-consuming and may therefore reduce the time that is available for other patient care duties.

It is an object of the present invention to improve the monitoring of medical technical apparatuses, in particular in rooms which are sensitive in terms of hygiene.

The solution of this object is provided by the teaching of the independent claims. Various embodiments and further developments of the invention are the subject of the dependent claims.

A first aspect of the invention relates to a method, in particular a computer-implemented method, for the remote monitoring of a medical technical apparatus used, in particular in a closed patient treatment room, for the medical monitoring or treatment of a specific patient, in particular from outside the patient treatment room, such as for example from an anteroom or corridor in front of it. The method is carried out by means of a mobile terminal apparatus which is separate from the medical technical apparatus and comprises: (i) detection, in a sensor-based manner, of a machine-readable patient identification assigned to the patient, in particular arranged in the local spatial environment of the patient; (ii) initiation of a data coupling between the medical technical apparatus and the terminal apparatus on the basis of the detected patient identification in order to establish a wireless, in particular bidirectional, communication link for the exchange of data assigned to the patient identification between the medical technical apparatus and the terminal apparatus; (iii) reception, via the communication link which has been established, of information transmitted by the medical technical apparatus to the terminal apparatus relating to the patient identified on the basis of the patient identification, or relating to their monitoring or treatment by the medical technical apparatus; and (iv) output of a representation, in particular an optical representation, e.g. a written and/or pictorial representation, or of an acoustic representation of the received information at a human-machine interface (HMI) of the terminal apparatus.

The “remote monitoring” of a medical technical apparatus (hereinafter also referred to as “apparatus” for short), in the sense of the invention, is to be understood to mean the control of the apparatus from the terminal apparatus, in which the apparatus and the terminal apparatus are wirelessly connected over a spatial distance (typically one meter or several meters) via a communication link, whereby information is exchanged between the apparatus and the terminal apparatus via the communication link, on the basis of which information, controlling, from the terminal apparatus, in the sense of monitoring or operation of the apparatus, is made possible with respect to at least one monitoring or operation aspect. The “remote controlling” of the apparatus from the terminal apparatus is thus a subtype of the remote monitoring, in which control data is transmitted from the terminal apparatus to the apparatus via the communication link, in order to control the apparatus in the sense of a configuration or any other operation.

A “mobile terminal apparatus”, in the sense of the invention, is to be understood to mean a portable data processing device, in particular a hand-held data processing device. It may in particular contain one or more processors and an information storage device coupled thereto and may be controlled by software. In particular, the terminal apparatus may be, or comprise, a handheld computer, in particular a “general purpose” computer, such as a tablet computer or a smartphone, configured in order to carry out the method, in particular by means of software. However, the use of terminal apparatuses which are specific to the application is equally possible.

A “machine-readable patient identification” is to be understood to mean an identification, in particular a code, which is individually assigned to a patient, which identification is provided in a machine-readable representation. Such a representation can be provided in particular optically by means of letters or numbers or other symbols, for example as a one-dimensional or multidimensional barcode (e.g. a data matrix code or a QR code). Such an optical representation may in particular be applied or otherwise formed on a substrate, such as for example labels made of paper or plastic, or may be displayed, in particular temporarily, on a display device, such as for example a screen. A machine-readable patient identification may also be provided as a signal, in particular as an electromagnetic signal (e. g. a radio signal) or as an acoustic signal, which can be detected by means of a corresponding sensor device and is in this way “machine-readable”. The machine-readable patient identification can be assigned to the respective patient, in particular in that it is arranged by way of a spatial assignment to the patient themselves, to their clothing, to a device associated with them, such as for example their patient bed, to a spatial area associated with them within their patient treatment room or at an access or anteroom to their patient treatment room.

A “data coupling” between the medical technical apparatus and the terminal apparatus on the basis of the detected patient identification is to be understood to mean a signal coupling, for example in contrast to a mechanical coupling, by means of which signal coupling a wireless communication link, in particular a bidirectional wireless communication link, for the exchange of data assigned to the patient identification is made available between the medical technical apparatus and the terminal apparatus. The coupling can in particular be of a temporary nature, whereby the duration of the coupling can be contingent on, in particular, the expiration of a predefined time interval, an active cancellation of the coupling or a replacement of the coupling by a coupling between the terminal apparatus and a different medical technical apparatus, or an interruption of the coupling due to loss, or excessive weakening, of the signal connection. The initiation of the data coupling between the medical technical apparatus and the terminal apparatus on the basis of the detected patient identification may in particular comprise the patient identification detected by the terminal apparatus being transmitted from the terminal apparatus to the medical technical apparatus. However, as an alternative, or in addition, it is in particular also possible that the medical technical apparatus itself receives the patient identification independently of the terminal apparatus, for example by means of its own detection or by a user input. In any case, on the basis of the patient identification which is now known to these two apparatuses, subsequently, a data exchange which is specifically related to this patient identification or the associated patient can thus be accomplished.

The wireless communication link may in particular be a radio communication link. For example, radio technology according to a known LAN (Local Area Network) or PAN (Personal Area Network) radio standard, such as for example WLAN (WIFI), Bluetooth or Zigbee, can be used for this purpose. In principle, however, other wireless communication links with a corresponding range are also conceivable, such as light-based, infrared-based or sound-based communication links.

With the help of the method mentioned above, a spatial separation between, on the one hand, the medical technical apparatus located by the patient and, on the other hand, the mobile terminal apparatus which is used for their remote monitoring can be achieved. With regard to the problems mentioned above regarding the burden in terms of hygiene and walking distances, this can be achieved in particular by the mobile terminal apparatus being used outside the patient treatment room in order to monitor, in the sense of a remote monitoring, via a wireless communication link, the medical technical apparatus which is located inside the patient treatment room. Thus, this monitoring can be performed by operating personnel, in particular by medical specialist personnel such as for example a doctor or a nurse, without there being a need for them to enter the area of the patient treatment room, which is sensitive in terms of hygiene, for which they would have to take the appropriate hygiene protection measures.

In case several medical technical apparatuses are to be monitored in the treatment room, this can be done by means of a corresponding successive coupling of the mobile terminal apparatus with a respective one of the medical technical apparatuses, in particular from outside the patient treatment room, without there being a need to cover the walking distances to the respective medical technical apparatuses within the patient treatment room. In addition, such walking distances can also be saved in situations in which, irrespective of whether this takes place in an environment which is particularly sensitive in terms of hygiene or whether this concerns a closed patient treatment room, monitoring of one or more medical technical apparatuses is to be carried out over a considerable distance, e.g. of at least a few meters.

In addition, the mobility of the terminal apparatus allows it to be used not only in connection with a specific medical technical apparatus or only a specific patient treatment room, but rather, several different operators, in particular doctors or health care personnel, may each have their own terminal apparatus which they can use for remote monitoring, in particular also remote control, of various suitable medical technical apparatuses in one or more different patient treatment rooms. In a similar vein, it is also possible for a specific medical technical apparatus to be monitored by different operators by means of their respective terminal apparatus within the framework of an ongoing patient monitoring or ongoing patient treatment, which is advantageous in particular with regard to shift changeover in hospital wards of a hospital or patient care by a patient care team comprising several persons.

In this way, the time and the hygiene measures required to monitor the medical technical apparatus or apparatuses can be reduced. In particular, this saved time and cost can be used to provide medical specialist personnel with more time to spend on other patient care.

The data coupling between the respective medical technical apparatus and the terminal apparatus on the basis of the patient identification assigned to the patient can therefore be used to ensure, in a quick and simple manner, that the monitoring which is carried out by means of the terminal apparatus relates to the correct patient and the correct medical technical apparatus monitoring or treating them, in particular in case several patients or apparatuses are present in the patient treatment room, so that incorrect treatment due to an incorrect assignment can be prevented in an effective manner.

In the following, preferred embodiments of the method are described, each of which, as far as this is not expressly excluded or technically impossible, can be combined in any desired manner with each other and with the other aspects of the invention described.

In some embodiments, the sensor-based detection of the patient identification comprises at least one of the following processes: (i) detection of a visual representation of the patient identification, in particular a representation of the patient identification on the basis of a one-dimensional or a multidimensional bar code, by means of an image sensor associated with the terminal apparatus, and automatic evaluation, in particular computer-aided evaluation, of the image data detected by the image sensor in order to determine the patient identification therefrom; (ii) detecting, by means of a radio receiver associated with the terminal apparatus, a radio signal representing the patient identification, and automatically evaluating, in particular in a computer-aided manner, the radio signal detected by the radio receiver in order to determine the patient identification therefrom.

The process (i) is characterized in particular by the fact that the representation of the patient identification can be produced very easily, for example by printing it on a substrate. The substrate can, for example, be a label, a tag, or a card for insertion into a card holder. In addition, or as an alternative, it is in particular also possible to display the representation of the patient identification by means of a display device, such as for example on a flat screen.

In the process (ii), the radio signal may in particular originate from a radio transmitter linked to the patient, for example a radio transponder. In this context, the transmission range of the radio transmitter is ideally limited to slightly more than the typical dimensions of a patient treatment room, for example a few meters. The radio solution according to process (ii) can be used to advantage in particular when lighting conditions are poor, especially at night, when any alternative optical detection of a patient identification by means of process (i) would possibly be impaired as a result. A further advantage is that, unlike the optical solution, the radio signal does not necessarily require a free line of sight to the representation of the patient identification or to the radio transmitter, so that a particularly high level of reliability and ease of use can be achieved here.

In some embodiments, the method further comprises: (i) image sensor-based detection of at least a portion of the patient and displaying of a representation of the image data generated in this way on the HMI of the terminal apparatus in such a way that the at least the partial image of the patient is displayed in the representation and marked as a selection by means of an Augmented Reality (AR) insertion; (ii) receipt of a user input on the HMI of the terminal apparatus for confirmation of the selection of the patient suggested by means of the marking. In this context, the initiation of the data coupling between the medical technical apparatus and the terminal apparatus on the basis of the detected patient identification is only carried out if the user input for confirming the selection of the patient has been received beforehand. In this way, a check is made in these embodiments in terms of whether the correct patient is selected. If, for example, two or more patients are present in a patient treatment room, it is in principle possible that, during the course of the detection of the patient identification, the patient identification of a patient other than the desired patient is incorrectly detected, which would lead to an incorrect assignment between the patient, the medical technical apparatus and the terminal apparatus. This situation can be avoided with the embodiments mentioned here by a check of the assignment on the basis of the image of the patient captured in an image sensor-based manner being carried out once again by a user of the terminal apparatus, whereby the AR marking indicates the current patient assignment. If the marked patient is the desired patient, the correctness of the assignment is confirmed by confirming the selection, whereas otherwise a correction can be made before the data coupling is initiated.

In some alternative embodiments, the sensor-based detection of a machine-readable patient identification associated with the patient further comprises a sensor-based detection of at least one further machine-readable patient identification which is associated with a different patient present in the treatment room, and the method further comprises: (i) image sensor-based detection of at least a portion of two or more of the patients whose patient identification has been detected in a sensor-based manner; (ii) displaying, on the HMI of the terminal apparatus, a representation of the image data generated during the course of the image sensor-based detection in such a way that in the representation, the respective at least partial image of the patients, that were detected in an image sensor-based manner, is contained and is marked by means of a respective, in particular different, such as for example a differently colored, Augmented Reality insertion; and (iii) reception of a user input on the HMI of the terminal apparatus for the selection of a particular one of the patients displayed on the HMI on the basis of the image data, on the basis of their respective marked image. In this context, the initiation of the data coupling between the medical technical apparatus and the terminal apparatus is carried out on the basis of the detected patient identification for the particular patient selected according to the user input. The patient identifications are preferably arranged respectively in the local spatial environment of the corresponding patient, for example on their patient bed or on the patient's body or clothing. These embodiments also serve to enable a verification of a correct patient assignment to the medical technical apparatus and to the terminal apparatus, whereby not only a confirmation but even a selection among different patients or patient identifications that have been recognized is provided here.

In some further developments of the embodiments mentioned above, for the confirmation or selection of a patient, the at least partial image sensor-based detection of the patient or patients comprises a respective automatic recognition of the patient or patients on the basis of the image data. In this context, the recognition of the patient or patients concerns at least a recognition of their image within the image represented by the image data. In this way, the AR marking can be formed, for example, as a frame around this patient image, which can run in particular along the outline of the patient image. In addition, the recognition can optionally also concern an identification of the patient or patients, which makes it possible in particular to compare these identifications with the machine-readable patient identification or identifications detected for the corresponding patient or patients, for the purpose of carrying out a consistency check. The latter can again be used in particular in order to increase the reliability of the assignment of a particular patient to a particular medical technical apparatus and to the terminal apparatus.

In accordance with some further embodiments, in some of these further developments mentioned above, the automatic recognition of the patient or patients on the basis of the image data is carried out in at least one of the following ways: (i) automatic pattern recognition in order to recognize the respective image of the patient within the image data; (ii) automatic identification of the respective patient on the basis of their machine-readable patient identification which is assigned and represented in the image data; (iii) automatic object recognition, within the image data, of at least one object or optical signal, such as for example the signal of a light source, which is uniquely assigned to the patient; (iv) automatic recognition, within the image data, of at least one AR marker which is spatially assigned to the patient. Common to all these possibilities is that they each enable reliable automatic recognition of the patient or patients on the basis of the image data.

The embodiments explained above regarding the recognition of a patient can be used analogously for the recognition of a medical apparatus (object) used for remote monitoring, in order to monitor or treat the selected patient. In particular, the apparatuses potentially to be detected can, for this purpose, each have an individual device identification, which is used analogously for the patient identification.

In some embodiments, the information which is received, at the terminal apparatus, from the medical technical apparatus, and which is output at the HMI of the terminal apparatus contains at least one of the following pieces of information: (i) an operating mode of the medical technical apparatus which is currently set on the terminal apparatus; (ii) a patient monitoring or patient treatment functionality which is currently running on the terminal apparatus; (iii) one or more actual measured parameters or currently set target operating parameters of the medical apparatus; (iv) one or more measured values relating to the physiological state of the patient monitored or treated by the medical technical apparatus; (v) one or more warnings relating to the current state or operation of the medical technical apparatus or the ongoing monitoring or treatment of the patient by means of the medical technical apparatus. In this way, the correct function of the medical technical apparatus or of a patient monitoring or patient treatment currently in progress or a current physiological state of the patient can be monitored from the terminal apparatus, in particular without there being a need for the medical personnel carrying out the monitoring to enter the patient treatment room, in particular by carrying out the corresponding, usually complex, hygiene protection measures.

In some embodiments, the output of the information received from the medical apparatus is configurable by the user and the method further comprises: (i) receiving a user input on the HMI of the terminal apparatus in order to determine a configuration regarding the selection or the display mode of the information to be output on the HMI; and (ii) configuring the terminal apparatus in such a manner that the information which from then on is received from the terminal apparatus is output, on the HMI, in accordance with the configuration determined by means of the user input with respect to its selection or display mode or both. In this way, the output of the received information on the HMI of the terminal apparatus can be arranged in a manner which is configurable by the user, which in particular enables a user-specific configuration, an application-specific configuration, a configuration which is specific to the medical technical apparatus used, or a patient-specific configuration. Thus, for example, for the patient monitoring or treatment of this patient currently in progress by means of the medical technical apparatus coupled to the terminal apparatus by means of data coupling, the state of the apparatus or of the patient that are critical specifically for this patient monitoring or treatment of this patient can be displayed preferentially on the HMI, for example in the uppermost display level of an operating menu specified by several display levels. By way of contrast, any information which is conversely irrelevant for this patient monitoring or treatment of this patient can for example be hidden or suppressed in order to avoid an overloading of the representation on the HMI and to make incorrect readings more difficult.

In some embodiments, the initiating of the data coupling between the medical technical apparatus and the terminal apparatus on the basis of the detected patient identification comprises establishing a bidirectional wireless communication link for the bidirectional data exchange of data associated with the patient identification between the medical technical apparatus and the terminal apparatus, and the method further comprises: (i) receiving a user input on the HMI of the terminal apparatus for remote control of the medical technical apparatus by means of the terminal apparatus; and (ii) transmitting control data via the communication link to the medical technical apparatus, which represent the remote control of the medical technical apparatus determined by means of the user input. In this way, not only a monitoring of the medical technical apparatus but, beyond that, also its control in the sense of a remote control from the terminal apparatus can be implemented. This can be used in particular to configure the medical technical apparatus, to respond to messages or signals from the medical technical apparatus to the terminal apparatus, for example to acknowledge messages, in particular warnings, or to directly control the medical technical apparatus, for example for the purpose of activating or deactivating the apparatus as a whole or of individual functionalities of the apparatus or to change treatment parameters.

In some further developments of these embodiments, if the control data, according to the user input, represents a change of a configuration or of a function of the medical terminal apparatus, such as for example an activation or a deactivation of a functionality thereof, the transmission of such control data only takes place if and when one or more of the following actions have been successfully carried out again by means of the terminal apparatus: (i) selection or confirmation of the patient being monitored or treated by means of the medical technical apparatus; (ii) authorization, in particular by authentication, of the user entering the user input for changing a configuration or a function of the medical terminal apparatus, for performing the corresponding remote control of the medical technical apparatus. In this way, the safety regarding the operation of the medical technical apparatus can be increased when it is operated by means of the terminal apparatus, in particular with a view to avoiding incorrect user input.

In some embodiments, the method further comprises a processing of the information received from the medical technical apparatus to automatically recognize one or more of the following: (i) potentially critical monitoring situations or treatment situations relating to the patient that is monitored or treated by the medical technical apparatus; (ii) potentially critical conditions of the medical technical apparatus; (iii) optimization potential regarding the operation of the medical technical apparatus, in particular in the context of the remote monitoring, in particular the remote control, of the apparatus from the terminal apparatus. With regard to variants (i) and (ii), this enables in particular a rapid and automatic detection of critical situations that potentially require an intervention by the medical personnel, without there being a need for a direct observation of the medical technical apparatus, or for information to be read from the medical technical apparatus itself. As far as such an intervention by the medical personnel is possible within the framework of the remote control of the medical technical apparatus, the critical situation that has been detected can also be remedied without there being a need for the burdensome entering of the patient treatment room, with the accompanying performance of the necessary hygiene measures, or at least minimizing walking distances. Additionally, variant (iii) makes it possible to automatically identify possibilities for the optimization of work processes on the basis of an automatic detection and, based thereon, evaluation of actual operating procedures carried out in relation to the medical technical apparatus. For example, if it is recognized in this way that, in connection with a dialysis machine, as a rule, too much time passes until an alarm that prompts the replacement of an almost empty bag is acted upon, a reconfiguration of the dialysis machine can be performed, in particular from the terminal apparatus, in such a way that in the future, such an alarm is triggered earlier, i.e. when the contents still remaining in the bag are at a higher level, in order to give the medical personnel more time to respond in good time.

According to some further developments of this, the processing of the information received from the medical technical apparatus for the automatic recognition of potentially critical situations regarding the monitoring or treatment or the recognition of potentially critical conditions of the medical technical apparatus can be carried out with the help of one or more data analysis methods using machine learning, for example by means of an artificial neural network. This enables a particularly flexible and adaptable automatic recognition of one or more of the various variants mentioned above, which can in particular also recognize situations, which would not be possible with conventional “rigid” analysis methods, in which the situations to be recognized are conclusively defined in advance.

In some embodiments, the method for the remote monitoring of the medical technical apparatus that is used for the medical monitoring or treatment of a specific patient is carried out while the medical technical apparatus is located in a closed patient treatment room and, at the same time, the mobile terminal apparatus is located outside the patient treatment room.

A second aspect of the invention relates to a mobile terminal apparatus for the remote monitoring of a medical technical apparatus used, in particular in a closed patient treatment room, for the medical monitoring or treatment of a specific patient, in particular from outside the patient treatment room, wherein the terminal apparatus is constructed to be separate from the medical technical apparatus and is configured to carry out the method according to the first aspect of the invention.

In some embodiments, the apparatus comprises in particular (i) a sensor device for the sensor-based detection of a patient identification provided locally by means of a device for providing an identification; (ii) a communication device for exchanging data with the medical technical apparatus; (iii) a human-machine interface, HMI, for detecting user inputs and for outputting information to a user of the terminal apparatus; and (iv) a data processing unit for the processing of user inputs detected via the HMI, of data received via the communication device and of information detected by means of the sensor device. The device for providing an identification can be constructed in particular as a display device, such as for example a screen, or as a signal transmitter, in particular a radio transmitter. It can also be provided, in particular, by means of printing on a substrate, as has already been described above. The substrate can for example be a label, a tag, or a card for insertion into a card holder.

Preferably, according to some of these embodiments, the communication device is configured for the wireless data exchange over a distance of, as a rule, no more than 15 m, preferably no more than 10 m. This can facilitate in particular the selective data coupling of the terminal apparatus with a specific medical technical apparatus, since due to the limited transmission range in cases of high apparatus density, as it is often the case in particular in hospitals, only those apparatuses are, from the outset, available for such a coupling which are available within the transmission range. In this way, the required transmission power can also be kept low, which can be used in particular to reduce or avoid undesired interference with other equipment caused by radio signals, as well as for spatially differentiated reuse of radio frequencies.

A third aspect of the invention relates to a computer program which is configured to run on a data processing unit of the terminal apparatus according to the second aspect of the invention and thereby to cause the terminal apparatus to execute the method according to the first aspect of the invention.

The computer program may in particular be stored on a non-volatile data carrier. This may in particular be a data carrier in the form of an optical data carrier or a flash memory module. This may be advantageous in particular if the computer program is to be traded as such, independently of a processor platform on which the one or more programs are to be run. In a further implementation, the computer program may be provided as a file on a data processing unit, in particular on a server, and may be available to download via a data connection, for example the Internet, or a dedicated data connection, such as for example a proprietary network or a local network, in particular to the terminal apparatus itself. In addition, the computer program may comprise a plurality of interacting, individual program modules.

In a corresponding manner, the terminal apparatus may comprise a memory storage, in which the computer program is stored. As an alternative, the terminal apparatus may also be set up to access a computer program that is available externally, for example one that is located on one or more servers or other data processing units, via a communication link, in particular to exchange data with the computer program, which data is used during the execution of the method or of the computer program or which represents outputs of the computer program.

A fourth aspect of the invention relates to a system for the monitoring or treatment of a patient. The system comprises: (i) a medical technical apparatus for the monitoring or treatment of the patient; (ii) an identification providing device for providing a machine-readable patient identification in the local environment of the patient or on the patient themselves, which patient identification is uniquely assigned to the patient; and (iii) a mobile terminal apparatus according to the second aspect of the invention. In this context, the terminal apparatus is configured to control the medical technical apparatus, in the sense of remote monitoring, in particular remote controlling, according to the method according to the first aspect of the invention, and the medical technical apparatus is configured to be controlled by the terminal apparatus in a corresponding manner.

In some embodiments of the system, the medical technical apparatus comprises at least one of the following medical technical functionalities: a dialysis functionality; a blood treatment functionality; an infusion pump functionality; a patient monitoring functionality. In particular in the case of apparatuses with one or more of these functionalities, it often happens that, on the one hand, they are used in an environment that is particularly protected in terms of hygiene by means of appropriate protective measures, in particular in patient treatment rooms that are particularly protected in terms of hygiene, and, on the other hand, that they must be monitored and/or operated by medical specialist personnel at high frequency. Therefore, the savings in terms of time and protection effort and thus also in terms of costs and waste disposal burden (especially for disposable protective clothing), which are made possible in accordance with the invention, are particularly effective here.

In some embodiments, the system further comprises a Wearable Electronics Device to be worn by the patient, wherein the Wearable Electronics Device comprises a memory storage device for storing data relating to the patient, in particular a patient identification assigned to the patient, as well as a communication device for wireless communication of data stored in the memory storage device to the mobile terminal apparatus or to the medical technical apparatus. This can in particular lead to the advantage that the data relating to the patient is directly carried (around) by the patient themselves and is thus always available at their current location without further action needing to be taken, one conceivable situation could involve a transfer of the patient from one patient treatment room to different one within the same hospital.

In some embodiments, the Wearable Electronics Device may in particular further comprise one or more of the following elements: (i) a security device in order to hinder an unauthorized removal of the Wearable Electronics Device from a patient wearing it; (ii) a signal device to emit an optical or acoustic signal in response to receiving a particular signal via the communication device. Variant (i) is primarily intended to make an unauthorized or accidental removal of the Wearable Electronics Device from the patient more difficult, ideally to prevent it, in order to avoid the burden necessary for reattachment. In case the Wearable Electronics Device is also used to accomplish the assignment of the patient to a medical technical apparatus monitoring or treating the patient and/or the terminal apparatus, which may in particular be the case if the Wearable Electronics Device is used to provide the patient identification of the patient, this variant (i) can also be used advantageously to secure the data coupling within the framework of the method according to the invention.

In some embodiments, the Wearable Electronics Device is constructed as one of the following: wrist strap, ankle strap, bracelet, ankle bracelet or necklace or lanyard; a sticker to be applied to the patient's skin or to their clothing, belt, or a part of their patient gown. All these embodiments allow, in particular, a reliable and mechanically robust attachment of the Wearable Electronics Device to the patient or their clothing without thereby significantly impairing the patient's well-being.

The features and advantages which have been explained in relation to the first aspect of the invention apply equally also to the other aspects of the invention.

Further advantages, features and possible applications of the present invention become clear from the following detailed description in connection with the figures.

In the figures:

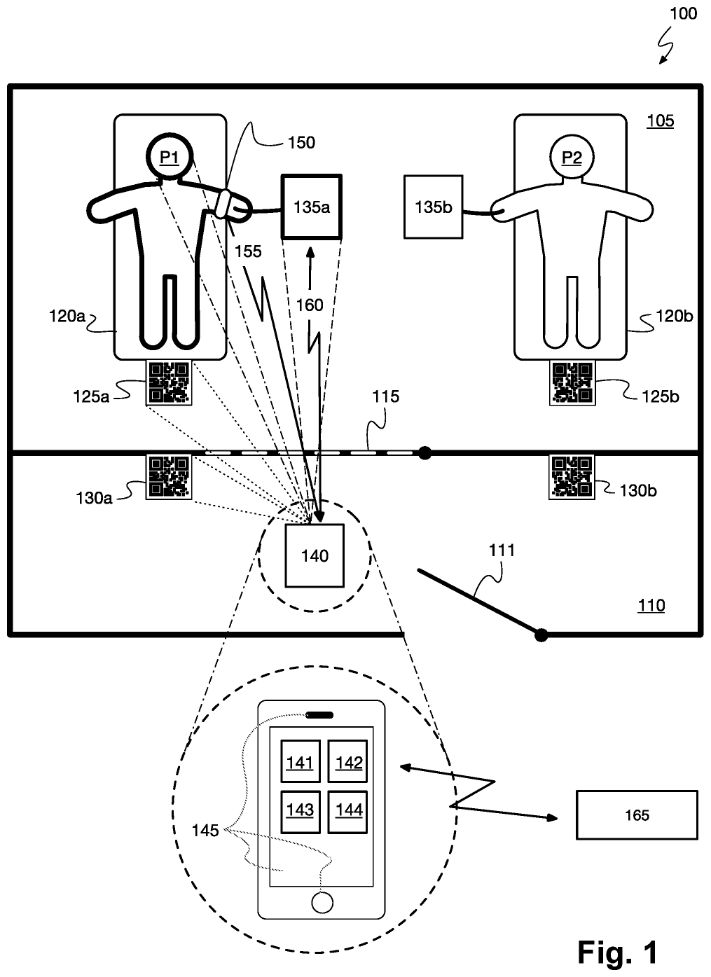

FIG. 1 shows a diagram of a hospital ward with a patient treatment room, together with an adjoining anteroom, as well as a system used in this environment, in accordance with an embodiment of the invention, for the monitoring or the treatment of patients present in the patient treatment room, whereby the system in particular comprises the terminal apparatus that is again shown enlarged, also in FIG. 1, at the bottom thereof; and

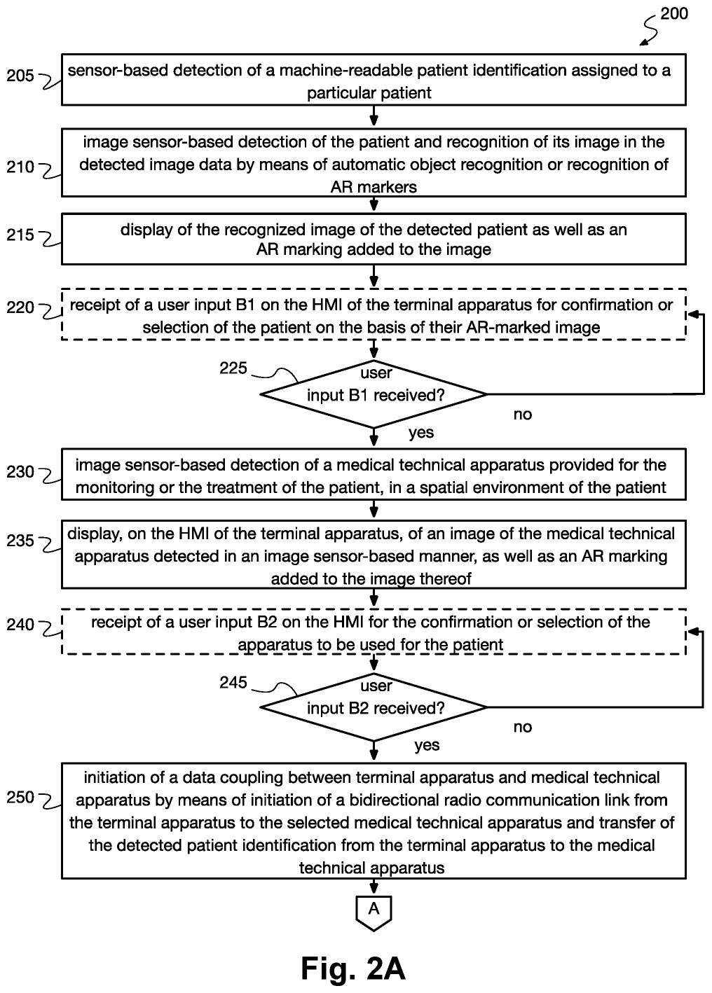

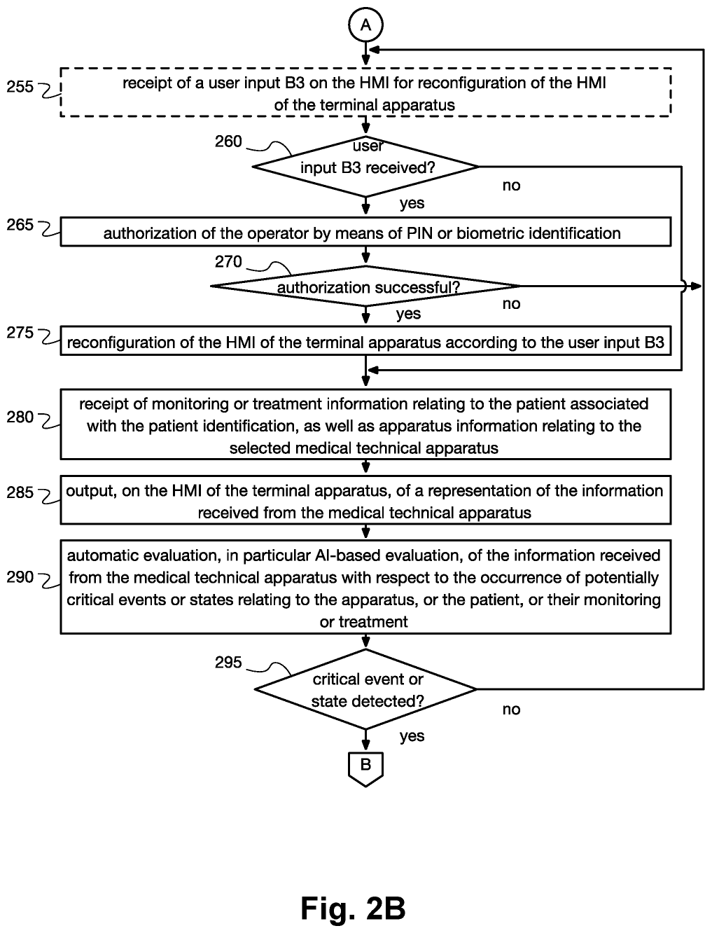

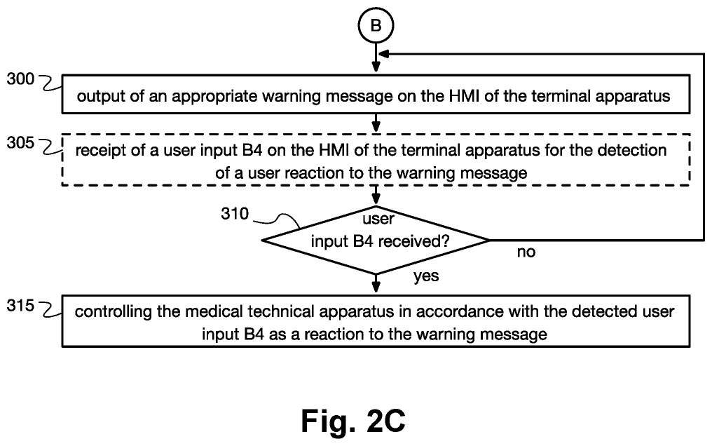

FIGS. 2A-C show a flowchart illustrating a preferred embodiment of the method according to the invention.

The hospital ward 100, shown in FIG. 1, comprises a patient treatment room 105, as well as an anteroom 110 which is located in front of it and which is linked via a connecting door 115, which serves as a hermetic barrier or airlock. The anteroom 110, in turn, comprises an outer door 111, through which it can be entered. The connecting door 115 has a viewing window (shown in dashed lines), through which the interior of the patient treatment room 105 can be seen from the anteroom 110. In the patient treatment room 105, there are two patients, P1 and P2, each on a patient bed, 120a and 120b respectively, which have been made available for them.

The connecting door 115 also serves to hygienically separate the patient treatment room 105, which is particularly sensitive in terms of hygiene, from the anteroom 110, which is subject to lower hygiene requirements. To enter the patient treatment room 105, protective clothing must typically be worn in order to maintain the required standard of hygiene, which protective clothing must be disposed of, by means of an appropriate disposal device 165, on the point of exiting the patient treatment room 105, before leaving the patient treatment room 105. In this way, the risk can be reduced that, on the one hand, germs might possibly be carried into the patient treatment room 105 by medical specialist personnel, and, on the other hand, that germs from the patients P1 and P2, which germs might possibly be present in the patient treatment room 105, could leave the room.

In situations in which, by way of contrast, no special hygiene protection measures are required, the connecting door can in particular be left out.

On each of the patient beds 120a and 120b a first device for providing an identification is provided, which displays a machine-readable first patient identification 125a or 125b, for example in the form of a QR barcode, which is uniquely assigned to the patient on the respective patient bed 120a or 120b, in such a way that it can be detected in an image sensor-based manner, in particular by means of a camera, from the anteroom 110 through the viewing window of the connecting door 115. In addition, the same patient identifications are again displayed by means of second devices for providing patient identifications as second patient identifications 130a or 130b in the anteroom 110, for example on the partition wall between the patient treatment room 105 and the anteroom 110 and, in this regard, ideally in spatial correspondence to the respective position of the patient bed 120a or 120b of the patient P1 or P2 associated with the respective patient identification.

The devices for providing patient identifications for the machine-readable patient identifications 125a or 125b and 130a or 130b can in particular be provided in the form of printed substrates, such as for example paper cards, but can also be provided by suitable display devices (e. g. screens) on the patient beds 120a or 120b or in the anteroom 110. The latter makes it possible, in particular, to flexibly replace the patient identifications displayed with a different one each time the occupancy of the patient treatment room 105 changes, without generating waste (substrates no longer required).

In addition, there are two medical technical apparatuses 135a and 135b located in the patient treatment room 105, each of which is individually assigned to the patients P1 and P2 in a one to one relationship. The apparatuses 135a 135b can in particular be dialysis machines or blood purification apparatuses or comprise a dialysis functionality or a blood purification functionality, among other things. Other medical technical functionalities are also equally possible. Each of the apparatuses 135a and 135b can be operated via a user interface provided on the respective apparatus. For direct operation of the respective apparatus, the medical specialist personnel must therefore enter the patient treatment room 105 and walk from the connecting door 115 to the respective apparatus 135a or 135b and back again, while observing the applicable hygiene regulations, in particular the wearing of protective clothing. The direct operation of the apparatuses 135a, b therefore involves a significant burden.

The patient P1 has a wrist strap 150 constructed as an electronics element which can be worn on the body (Wearable Electronics Device), which is attached to his or her arm and which is secured against unintentional and unauthorized removal, for example in such a way that it can only be removed by means of a tool. The wrist strap 150 comprises a radio transmitter which is configured to transmit the individual patient identification previously assigned to the patient P1, which corresponds to that shown in the bar codes 125a and 130a, by means of a radio signal 155. The wrist strap 150 has a memory storage means in which, in addition to the patient identification, other data relating to the patient (e. g. biographical information, medical protocols, treatment history, etc.) can be stored as well, so that this data is available via the radio signal 155 at the terminal apparatus 140 and/or at the apparatus 135a. The wrist strap can also be equipped with optical or acoustic signaling means. Instead of a wrist strap, embodiments in particular in the form of a belt, a lanyard or a sticker that can be positioned variably on the patient are also conceivable.

In addition to the devices for providing a patient identification 125a, b and 130a, b, and the medical technical apparatuses 135a, 135 as well as the wrist strap 150, the embodiment of a system in accordance with the invention shown (inter alia) in FIG. 1 also includes a mobile terminal apparatus 140, which can be constructed in particular as a tablet computer or a smartphone with corresponding programming for carrying out the method in accordance with the invention. Instead of this, an application-specific terminal apparatus is also possible.

The terminal apparatus 140 comprises an image sensor 141 constructed as a camera, a communication device 142, a processor, in particular an application processor, 143 as well as a, preferably non-volatile, memory device 144 for storing data and computer programs. In particular, the method 200 can accordingly take the form of a computer-implemented method and can be implemented or controlled by a corresponding computer program stored in the memory device 144.

In addition, the terminal apparatus 140 is equipped with a human-machine interface (HMI) 145, which, in the case of a tablet computer or smartphone, in particular comprises, as is known, a touch-sensitive screen and optionally one or more real buttons or switches. The medical technical apparatuses 135a and 135b are each set up to communicate with the communication device 142 of the terminal apparatus 140, preferably bidirectionally, via a wireless communication link in case a data coupling with the terminal apparatus 140 has been established. FIG. 1 illustrates the case where the terminal apparatus 140 is connected to the medical technical apparatus 135a in a data-coupling manner with respect to the patient identification of the patient P1, and a wireless communication link 160 exists between the two.

In the same way, the terminal apparatus 140 could however alternatively also be coupled to the medical technical apparatus 135b of the patient P2 with regard to this patient's patient identification, as shown for example by barcode 125b. In fact, the terminal apparatus 140 can be selectively, and in particular temporarily, alternatively be connected to one or the other of the medical technical apparatuses 135a or 135b in a data-coupling manner in order to thereby exchange data assigned to the respective patient P1 or P2 on the basis of their respective patient identification.

In addition, the terminal apparatus 140 can enter into a communication link, in particular a bidirectional communication link, with an external server or a computer network 165 via a corresponding data communication link. The terminal apparatus can in particular also enter into, or be in, communication with other comparable terminal apparatuses via this server or computer network. This enables the invention to be combined with other common applications on the basis of data communication, such as for example instant messenger (for text/voice communication with other members of staff), time synchronization (time, work planning, appointments, etc.), remote authorization of steps of treatment, automatic archiving of data and user behavior processed by the terminal apparatus, speech recognition and voice [symbol] control of the terminal apparatus, and provision of training materials related to treatment.

In the following, an example embodiment 200 of the method in accordance with the invention, carried out by the terminal apparatus 140, will be explained, with reference to the flowchart shown in FIGS. 2A to 2C, which are interconnected by means of the connectors A and B, whereby for the purpose of a better illustration, reference will additionally also be made to the arrangement which has already been explained and which is shown in FIG. 1.

In the method 200, which, in the following, will be explained with reference to the patient P1 as the selected patient by way of example, the sensor-based detection of a machine-readable patient identification assigned to the patient P1 takes place in a step 205. In the present example, according to FIG. 1, this can selectively be either one of the patient identifications 125a or 130a, respectively represented as a QR code, or alternatively, the patient identification which is transmitted by means of the radio signal 155 from the wrist strap 150 to the terminal apparatus 140, whereby the patient identifications mentioned correspond to one another. For the detection of one of these patient identifications, the terminal apparatus 140 comprises an image sensor 141, in particular a camera, and a radio communication device 142. The process of detecting one of the patient identifications is typically performed by a user—usually this will be a member of medical staff, such as a doctor or a nurse—operating and holding the terminal apparatus 140 in such a way that the patient identification 125a or 130a is within the field of view of the image sensor 141 and can thus be read by the terminal apparatus 140, or in such a way that the radio signal 155 can be received by the radio communication device 142.

In a further step 210, the patient P1 is detected in a sensor-based manner through the viewing window of the connecting door 115 by means of the image sensor 141 of the terminal apparatus 140, whereby corresponding image data is generated. In addition, the image data is analyzed by means of automatic object recognition in order to identify an outline of the image of the patient P1 in the captured image. In addition, or as an alternative, the image of the patient P1 can also be approximately identified by recognizing one or more Augmented Reality (AR) markers which are attached to the patient or their clothing (not shown). For this purpose, the image captured in real time by the image sensor 141 is displayed on the terminal apparatus and scanned for trigger features. These features correspond to the usual triggers for “Augmented Reality” (AR) applications, e.g. visible markers (stickers) on the apparatuses and/or beds, GPS positions of the objects filmed. The triggering can also be carried out by intelligent object recognition in the image and/or by object identification on the basis of the position of the terminal apparatus 140 relative to the object (here: the patient P1) in a virtual space. Then, in the step 215, the previously recognized image of the patient P1 detected in an image sensor-based manner is visually displayed on the HMI 145 of the terminal apparatus 140, whereby the recognized outline is highlighted by means of an AR mark, for example by means of a colored frame. In FIG. 1, this is illustrated, by way of example, by means of the outline of the patient P1 being displayed with a larger line width when compared with that of patient P2.

Now the terminal apparatus 140 waits for the receipt of a user input B1 on the HMI 145 in a subsequent step 220 for the purpose of confirming or selecting the detected patient P1. This confirmation or selection can be carried out in particular by the user touching the area delimited by the AR mark on a touch screen of the HMI and this accordingly being detected by the terminal apparatus 140 and interpreted as a confirmation or selection of the patient P1.

If, and as soon as, the user input B1 has been received on the HMI 145 and this user input B1 has been correctly interpreted as a confirmation or selection of the patient P1 belonging to the patient identification detected in the step 205 (225—yes), the terminal apparatus 140 is ready to detect, in a further step 230, in an image sensor-based manner, a medical technical apparatus which is to be used for the monitoring or the treatment of the selected patient P1. In the given example of FIG. 1, this is the apparatus 135a which is located in the immediate vicinity of the patient P1. For the purpose of this image sensor-based detection, the terminal apparatus 140 is again held by the user with the appropriate orientation so that the image sensor-based detection of the apparatus 135a can be carried out from the anteroom 110 along a line of sight through the viewing window of the connecting door 115.

Then, in a further step 235, an image of the medical technical apparatus 135a detected in an image sensor-based manner is displayed on the HMI 145 of the terminal apparatus, whereby, in a manner comparable to step 210, for the patient P1, here in step 235 an outline of the image of the terminal apparatus 135a is recognized in the image data detected in step 230, and this outline is displayed on the HMI 145 by means of an AR mark in the displayed image. If, in the course of the image sensor-based detection in step 230, the additional apparatus 135b was also detected and recognized as a potentially selectable medical technical apparatus in the context of the object recognition, this apparatus is additionally displayed on the HMI 145 together with a different AR mark, in particular a differently colored AR mark. In analogy to the steps 220 and 225, the reception of a user input B2 on the HMI 145 is then awaited in the steps 240 and 245, for the confirmation or selection of the apparatus 235a for use with the patient P1, which apparatus 235a has an AR mark on the HMI 145. The personnel can select a specific one of the two apparatuses 135a, b, by input on the terminal apparatus 140, as a result of which this selected apparatus is preferably focused, i.e. a change of the captured image section (or a change of the position of the terminal apparatus) no longer changes the displayed position of the apparatus on the HMI 145.

If, and as soon as, the user input B2 has been received on the HMI 145 and this user input B2 has been correctly interpreted as a confirmation or a selection of the medical technical apparatus to be used for the monitoring or the treatment of the previously selected patient P1 (245—yes), there follows a step 250, in which a data coupling is initiated between the terminal apparatus 140 and the selected medical technical apparatus (in this case this is chosen to be the apparatus 235a). The initiation of the data coupling between the medical technical apparatus 235a and the terminal apparatus 140, on the basis of the previously detected patient identification of the patient P1, involves establishing a bidirectional wireless communication link 160 for the bidirectional data exchange, between the medical technical apparatus 135a and the terminal apparatus 140, of data associated with the patient identification of the patient P1.

In particular, for this purpose, the patient identification of the patient P1, which has been detected in step 205, is transmitted from the terminal apparatus 140 to the selected apparatus 135a via the communication link 160, which has been established. In addition, or as an alternative, the apparatus 135a can also detect this patient identification itself, for example by receiving the radio signal from the wrist strap 150 or by scanning the barcode 125a. The deciding factor is that both the medical technical apparatus 135a as well as the terminal apparatus 140 know the patient identification of the patient P1 and that they exchange data, within the framework of the data coupling, which data relates exclusively to the associated patient P1, in particular to their monitoring or treatment by the apparatus 135a, or relates to the apparatus 135a itself, but not, for example, to the other patient P2 or to another medical technical apparatus used for their care (in the example from FIG. 1, this is the apparatus 135b).

The successful establishment of the data coupling is confirmed visually on the HMI 145, e.g. through the AR marking of the apparatus 135a already mentioned, and on the apparatus 135a itself, e.g. through an illumination of the screen of the apparatus and/or through light sources attached to the patient bed 120a of P1. For this purpose, operating data is transmitted wirelessly (via common encrypted wireless communication, e.g. Wi-Fi) from the terminal apparatus 140 to the apparatus 135a. This confirmation ensures that the operating personnel can visually identify the selected apparatus 135a from a distance (and, if applicable, through a glass pane) in an unambiguous manner.

The data coupling established in step 250 between the terminal apparatus 140 and the medical technical apparatus 135a is typically of a temporary nature and, as a rule, is maintained for as long as the wireless communication link 160 exists or until the terminal apparatus 140 is instead linked in a data coupling manner to a different medical technical apparatus, for example the apparatus 135b for the care of patient P2. In this way, it is possible to use one and the same terminal apparatus 140 for the remote monitoring of different medical apparatuses, in particular also for the care of different patients. In order to avoid treatment errors, the terminal apparatus 140 is preferably linked by data coupling, in the manner described above, to no more than one medical technical apparatus at any given point in time, although alternative embodiments, in which a data coupling to different medical technical apparatuses exist simultaneously, which are displayed on the HMI, for example in different windows or menu levels, are also possible.

However, the HMI 145 of the terminal apparatus 140 can also have a configuration option for the user. In principle, such a configuration or reconfiguration can be performed at different points in time during operation of the terminal apparatus 140. However, since a reconfiguration of the HMI 145 is in particular useful in connection with the selection of a specific medical technical apparatus, it is now described in the present example starting with the next step 255 of the method, in which a corresponding user input B3 to activate a reconfiguration mode and to define a reconfiguration of the HMI 145 is received at the terminal apparatus 140. The user input B3 can accordingly comprise several individual inputs. The same also applies to the other user inputs B1, B2 and B4 described in connection with the FIGS. 2A to 2C. The reconfiguration defined by means of the user input B3 may relate in particular to the selection of the information or control elements to be displayed, or the way in which they are represented on the HMI 145.

If, and as soon as, the user input B3 has been received (260—yes), the operator gains authorization in the form of an authentication, which can be done in particular by entering a PIN on the HMI 145 or by means of biometric identification, for example by means of a fingerprint sensor or a face or iris recognition on the terminal apparatus 140. If the authorization fails (270—no), for example due to the entry of an incorrect PIN, the method branches back to the step 255. Otherwise (270—yes), the terminal apparatus 140 reconfigures its HMI 145 in step 275 in accordance with the user input B3 and then continues to the subsequent step 280. If, on the other hand, it is determined in the step 260 that the reconfiguration mode has not been initiated and accordingly that no user input P3 has been received for reconfiguration (260—no), the steps 265 to 275 are skipped and the method continues directly with the step 280. Instead of this, the authorization may also be provided at an earlier stage of the method, in particular also at the beginning of the method.

Once the identification of the patient P1, the apparatus 135a and the personnel has been successfully carried out in this way, a signal that can be noticed by staff (e. g. a sound or an illumination of the screen of the apparatus or other light sources close to the patient), can optionally be emitted at the apparatus 135a, which signals that the apparatus 135a is ready to receive changes, which are to be entered on the terminal apparatus 140. The terminal apparatus 140 is then able to transfer control data to the apparatus 135a that is to be controlled remotely. When the data coupling is established, the terminal apparatus 140 receives, from the previously selected apparatus 135a, data associated with the patient P1 or their associated patient identification, which data may in particular contain information relating to their monitoring or their treatment and/or information relating to the apparatus 135a itself. For this purpose, the entire amount of data which has been collected by the apparatus 135a is potentially available for selection. In the first instance, this is typically data that is (or can be) also displayed at the user interface of the apparatus itself. This does not necessarily have to be data exclusively related to treatment. Such values may concern, in particular with regard to dialysis or blood purification, for example a current treatment option, (blood) pressure, pulse, (body/fluid/machine component) temperatures, patient data (ID, name, age, sex, medical protocols, etc.), patient weight, (treatment/running) times, parameters relating to the effectiveness of the dialysis (clearance, Kt/V), (ultrafiltration) volumes, dialysis filters/tubes/solvents (solvent compositions) used, flow rates, pump rates, or electrical voltages, as well as information about the machine (e.g., serial number, software version, machine type, GPS position, connected equipment). The subset of data to be displayed can be correlated with access rights of medical personnel determined in advance.

The information which has been received in the step 280 is then output at the HMI 145 of the terminal apparatus 140 in step 285, in accordance with the configuration of the HMI, which may have been previously defined. This can take place in a variety of ways, for example by means of one or more symbols, graphics, or as a numeric or written output. In addition, it is also possible to display other information, such as for example information generated by the terminal apparatus 140 itself or further information transmitted by the apparatus 135a.

The information received can also be automatically evaluated in a further step 290, which can be done in particular by means of one or more methods from the field of artificial intelligence, in particular those based on machine learning. In this way, certain information patterns in particular, which point towards the occurrence of potentially critical events or states with regard to the apparatus or the patient or the patient's care can be detected flexibly and in a manner which allows their detection to be continuously improved. If, for example, one or more of the medical values mentioned above (e.g. blood pressure, pulse) reaches a critical range, the sounding of an alarm at the terminal apparatus can be triggered, as a rule, at the medical technical apparatus 135h as well.

In addition, by means of such an automatic evaluation, a potential for improvement in the use of the apparatus 135a or the care of patient P1 as a whole can possibly be opened up. In the case of a dialysis, for example, it could be recognized that bags are exchanged very late too often, shortly before they become empty, and therefore a potential for improvement can be identified here, which can be addressed in particular by an appropriately early alarm at the terminal apparatus 140 or in another way. In order for the evaluation to be carried out, it is optionally also possible to make use of the server 165 and for the evaluation to be outsourced to it, at least partially, which makes it possible to use its computing power, which, as a rule, is higher than that of the mobile terminal apparatus and thus to carry out the evaluation quicker. This also makes it possible to implement a more effective and faster machine learning centrally, on the basis of the information received from several terminal apparatuses.

A possible objective, and in particular one that is relevant in the context of intensive care, which is to be achieved by such evaluations, primarily serves to provide support to staff and to patients in everyday clinical practice. To be mentioned as potential effects of the invention in this context are, for example, faster/better reaction to critical situations, better patient safety, better hygiene, reduction of workload of the staff, lower consumption of materials, fewer operating errors, reduction of the risk of accidents, etc.

If no critical events and no critical conditions are detected within the framework of the automatic evaluation in step 290 (295—no), the method branches back to before the step 255 or, optionally (not shown), to before the step 280. Otherwise (295—yes), warning signals are issued at the HMI 145 of the terminal apparatus 140, for example in an audible manner and/or by means of a corresponding display, which warning signals correspond to the potentially critical event(s) or condition(s) which have been detected. As a rule, corresponding warning signals are also issued at the medical technical apparatus 135a itself.

The terminal apparatus 140 then waits for the receipt of a user input B4 in order to detect a user reaction to the warning signal or signals issued. If, and as soon as, a corresponding user input B4 has been received at the HMI 145 in a step 305 (310—yes), by means of which for example the warning has been acknowledged or another control command for controlling the medical technical apparatus 135a has been entered, the medical technical apparatus 135a is then controlled in the step 315 in the sense of a remote control according to the detected user inputs B4. In this way, the warning signal can in particular be switched off or muted, for example, or the function of the apparatus 135A can be controlled in another way at both the apparatus 135a as well as at the terminal apparatus 140 itself as a response to the warning message.

Of course, it is also possible to control the medical technical apparatus 135a independently of a warning message being received first, by means of a corresponding control input on the HMI 145 of the terminal apparatus, as long as the data coupling between the two apparatuses exists. The implementation of the changes made to the apparatus 135a by remote control or reconfiguration via the terminal apparatus 140 can also be acknowledged, in particular again by a corresponding signal output at the apparatus 135a and optionally also at the HMI 145 of the terminal apparatus 140 itself.

While at least one example embodiment has been described above, it should be noted that there are a large number of variations to this. It is also to be noted that the embodiments described by way of example only represent non-limiting examples, and it is not intended to thereby limit the scope, the applicability or the configuration of the devices and methods described here. Rather, the preceding description will provide the skilled person with instructions for the implementation of at least one example embodiment, whereby it is to be understood that various changes may be made in the functionality and arrangement of the elements described in an example embodiment without departing from the subject matter respectively defined in the appended claims, as well as its legal equivalents.

### LIST OF REFERENCE SIGNS

100 hospital ward with patient treatment room including anteroom and remote monitoring system

105 patient treatment room

110 anteroom of the patient treatment room

111 outside door of the anteroom

115 connecting door with viewing window

120a, b patient beds

125a, b first devices for providing identifications or first machine-readable patient identifications in the form of QR codes attached to the patient beds

130a, b second devices for providing identifications or second machine-readable patient identifications in the form of QR codes installed in the anteroom

135a, b medical technical apparatuses, in particular dialysis machines or blood purification apparatuses

140 mobile terminal apparatus, in particular a tablet computer or a smartphone

141 image sensor, in particular a camera

142 radio communication device

143 processor

144 memory device

145 human-machine interface, HMI of the terminal apparatus 140

150 Wearable Electronics Device in the form of a patient wrist strap with a radio transmitter for patient identification

155 wireless communication link between the Wearable Electronics Device 150 and the terminal apparatus 140

160 wireless communication link between apparatus 135a and terminal apparatus 140

165 server

200 embodiment of a method in accordance with the invention

205-315 method steps of the method 200

B1-B4 user inputs on the HMI 145

P1 first patient

P2 second patient

1. A method for the remote monitoring of a medical technical apparatus used for the medical monitoring or treatment of a specific patient, wherein the method is carried out by means of a mobile terminal apparatus which is separate from the medical technical apparatus and wherein the method comprises:
detection, in a sensor-based manner, of a machine-readable patient identification assigned to the patient;
initiation of a data coupling between the medical technical apparatus and the terminal apparatus on the basis of the detected patient identification, in order to establish a wireless communication link for the exchange of data assigned to the patient identification between the medical technical apparatus and the terminal apparatus;
reception, via the communication link which has been established, of information transmitted by the medical technical apparatus to the terminal apparatus relating to the patient identified on the basis of the patient identification, or relating to their monitoring or treatment by the medical technical apparatus; and
output a representation of the received information at a human-machine interface, HMI, of the terminal apparatus.

2. The method according to claim 1, wherein the sensor-based detection of the patient identification comprises at least one of the following processes:
detection of a visual representation of the patient identification by means of an image sensor associated with the terminal apparatus, and automatic evaluation of the image data detected by the image sensor in order to determine the patient identification therefrom;
detecting, by means of a radio receiver associated with the terminal apparatus, a radio signal representing the patient identification, and automatically evaluating the radio signal detected by the radio receiver in order to determine the patient identification therefrom.

3. The method according to claim 1, further comprising:
image sensor-based detection of at least a portion of the patient and displaying of a representation of the image data generated in this way on the HMI of the terminal apparatus in such a way that the at least the partial image of the patient is displayed in the representation and marked as a selection by means of an Augmented Reality insertion;
receipt of a user input on the HMI of the terminal apparatus for confirmation of the selection of the patient suggested by means of the marking;
wherein the initiation of the data coupling between the medical technical apparatus and the terminal apparatus on the basis of the detected patient identification is only carried out if the user input for confirming the selection of the patient has been received beforehand.

4. The method according to claim 1, wherein the sensor-based detection of a machine-readable patient identification associated with the patient further comprises a sensor-based detection of at least one further machine-readable patient identification which is associated with a different patient present in the treatment room; and
the method further comprises:
image sensor-based detection of at least a portion of two or more of the patients whose patient identification has been detected in a sensor-based manner;
displaying, on the HMI of the terminal apparatus, a representation of the image data generated during the course of the image sensor-based detection in such a way that in the representation the respective at least partial image of the patients, that were detected in an image sensor-based manner, is contained and is marked by means of a respective Augmented Reality insertion; and
reception of a user input on the HMI of the terminal apparatus for the selection of a particular one of the patients displayed on the HMI on the basis of the image data, on the basis of their respective marked image;
wherein the initiation of the data coupling between the medical technical apparatus and the terminal apparatus is carried out on the basis of the detected patient identification for the particular patient selected according to the user input.

5. The method according to claim 3, wherein the at least partial image sensor-based detection of the patient or patients comprises a respective automatic recognition of the patient or patients on the basis of the image data.

6. The method according to claim 5, wherein the automatic recognition of the patient or patients on the basis of the image data is carried out in at least one of the following ways:
automatic pattern recognition in order to recognize the respective image of the patient within the image data;
automatic identification of the respective patient on the basis of their machine-readable patient identification which is assigned and represented in the image data;
automatic object recognition, within the image data, of at least one object or optical signal which is uniquely assigned to the patient;
automatic recognition, within the image data, of at least one AR marker which is spatially assigned to the patient.

7. The method according to any one of the preceding claims claim 1, wherein the information which is received, at the terminal apparatus from the medical technical apparatus and which is output at the HMI of the terminal apparatus contains at least one of the following pieces of information:
an operating mode of the medical technical apparatus which is currently set on the terminal apparatus;
a patient monitoring or patient treatment functionality which is currently running on the terminal apparatus;
one or more actual measured parameters or currently set target operating parameters of the medical apparatus;
one or more measured values relating to the physiological state of the patient monitored or treated by the medical technical apparatus;
one or more warnings relating to the current state or operation of the medical technical apparatus or the ongoing monitoring or treatment of the patient by means of the medical technical apparatus.

8. The method according to claim 1, wherein the output of the information received from the medical apparatus is configurable by the user and wherein the method further comprises:
receiving a user input on the HMI of the terminal apparatus in order to determine a configuration regarding the selection or the display mode of the information to be output on the HMI; and
configuring the terminal apparatus in such a manner that the information which from then on is received from the terminal apparatus is output, on the HMI, in accordance with the configuration determined by means of the user input with respect to its selection or display mode or both.

9. The method according to claim 1, wherein the initiating of the data coupling between the medical technical apparatus and the terminal apparatus on the basis of the detected patient identification comprises establishing a bidirectional wireless communication link for the bidirectional data exchange of data associated with the patient identification between the medical technical apparatus and the terminal apparatus, and wherein the method further comprises:
receiving a user input on the HMI of the terminal apparatus for remote control of the medical technical apparatus by means of the terminal apparatus; and
transmitting control data via the communication link to the medical technical apparatus which represent the remote control of the medical technical apparatus determined by means of the user input.

10. The method according to claim 9, wherein, if the control data, according to the user input, represents a change of a configuration or of a function of the medical terminal apparatus, the transmission of such control data only takes place if and when one or more of the following actions have been successfully carried out again by means of the terminal apparatus:
selection or confirmation of the patient being monitored or treated by means of the medical technical apparatus;
authorization of the user entering the user input for changing a configuration or a function of the medical terminal apparatus, for perform the corresponding remote control of the medical technical apparatus;

11. The method according to claim 1, further comprising:
processing of the information received from the medical technical apparatus to automatically recognize one or more of the following:
potentially critical monitoring situations or treatment situations relating to the patient that is monitored or treated by the medical technical apparatus;
potentially critical conditions of the medical technical apparatus;
optimization potential regarding the operation of the medical technical apparatus.

12. The method according to claim 11, wherein the processing of the information received from the medical technical apparatus for the automatic recognition of potentially critical situations regarding the monitoring or treatment or the recognition of potentially critical conditions of the medical technical apparatus is carried out with the help of one or more data analysis methods using machine learning.

13. The method according to claim 1, wherein the method for the remote monitoring of the medical technical apparatus that is used for the medical monitoring or treatment of a specific patient is carried out while the medical technical apparatus is located in a closed patient treatment room and, at the same time, the mobile terminal apparatus is located outside the patient treatment room.

14. A mobile terminal apparatus for the remote monitoring of a medical technical apparatus used for the medical monitoring or treatment of a specific patient, wherein the terminal apparatus is constructed to be separate from the medical technical apparatus and is configured to carry out the method according to claim 1.

15. The mobile terminal apparatus according to claim 14, comprising:
a sensor device for the sensor-based detection of a patient identification provided locally by means of a device for providing an identification;
a communication device for exchanging data with the medical technical apparatus;
a human-machine interface, HMI, for detecting user inputs and for outputting information to a user of the terminal apparatus; and
a data processing unit for the processing of user inputs detected via the HMI, of data received via the communication device and of information detected by means of the sensor device.

16. The mobile terminal apparatus according to claim 15, wherein the communication device is configured for the wireless data exchange over a distance of, as a rule, no more than 15 m.

17. A computer program which is configured to run on a data processing unit of the a terminal apparatus and thereby to cause the terminal apparatus to execute the method of claim 1, wherein the terminal apparatus is constructed to be separate from the medical technical apparatus and is configured to carry out the method.

18. A system for the monitoring or treatment of a patient, wherein the system comprises:
a medical technical apparatus for the monitoring or treatment of the patient;
an identification providing device for providing a machine-readable patient identification in the local environment of the patient or on the patient themselves, which patient identification is uniquely assigned to the patient; and
a mobile terminal apparatus;
wherein the terminal apparatus is configured to control the medical technical apparatus in the sense of remote monitoring, in accordance with the method of claim 1, and the medical technical apparatus is configured to be controlled by the terminal apparatus in a corresponding manner, wherein the mobile terminal apparatus is constructed to be separate from the medical technical apparatus and is configured to carry out the method.

19. The system according to claim 18, wherein the medical technical apparatus comprises at least one of the following medical technical functionalities:
a dialysis functionality; a blood treatment functionality; an infusion pump functionality; a patient monitoring functionality.

20. The system according to claim 18, further comprising a Wearable Electronics Device to be worn by the patient, wherein the Wearable Electronics Device comprises a memory storage device for storing data relating to the patient, as well as a communication device for wireless communication of data stored in the memory storage device to the mobile terminal apparatus or to the medical technical apparatus.

21. The system according to claim 20, wherein the Wearable Electronics Device further comprises one or more of the following elements:
a security device in order to hinder an unauthorized removal of the Wearable Electronics Device from a patient wearing it;
a signal device to emit an optical or acoustic signal in response to receiving a particular signal via the communication device.

22. The system according to claim 20, wherein the Wearable Electronics Device is constructed as one of the following: wrist strap, ankle strap, bracelet, ankle bracelet or necklace or lanyard; a sticker to be applied to the patient's skin or to their clothing, belt, or a part of their patient gown.
